(19) United States
(12) Patent Application Publication
(10) Pub. No.: US 2004/0260244 A1
(43) Pub. Date: Dec. 23, 2004

(54) Seals for trocars

(76) Inventors: Michael E. Piechowicz, Marcy, NY (US); Daniel M. Wing, Utica, NY (US)

Correspondence Address: HESLIN ROTHENBERG FARLEY & MESITI PC, 5 COLUMBIA CIRCLE, ALBANY, NY, 12203, US

(21) Appl. No.: 10/839,776

(22) Filed: May 5, 2004

Related U.S. Application Data
(63) Continuation-in-part of application No. 10/264,530, filed on Oct. 4, 2002.
Application No. 10/264,530 is a continuation-in-part of application No. 09/944,190, filed on Aug. 31, 2001.

(51) Int. Cl. A61M 5/178

(52) U.S. Cl. 604/167.06; 606/167

(57) ABSTRACT

A seal such as a releasably attachable upper seal for a trocar includes a rigid distal support, a rigid proximal support, a rotatable flexible seal having upper portions sandwiched between the supports, a movable ring disposed between the flexible seal and the lower support, and a generally non-compliant, but bendable protective guide attached to the inside of seal. The opening in the ring is sized smaller than the opening in the lower support. Cannulas and trocar systems employing such a seal are also disclosed.

### CROSS-REFERENCE TO RELATED APPLICATIONS

This application is a continuation-in-part of U.S. patent application Ser. No. 10/264,530, filed Oct. 4, 2002, entitled “Trocar System,” which is a continuation-in-part of U.S. patent application Ser. No. 09/944,190, filed Aug. 31, 2001, entitled “Obturator and Cannula For A Trocar Adapted For Ease Of Insertion And Removal,” the entire subject matter of these applications are incorporated herein by reference.

### TECHNICAL FIELD

This invention relates generally to surgical instruments, and more for particularly to seals for trocar systems for providing an opening through tissue and into body cavities and through which surgical instruments may be inserted.

### BACKGROUND OF THE INVENTION

Trocar systems are surgical devices used to obtain access to a body cavity to perform various surgical procedures such as laparoscopic surgery or arthroscopic surgery.

A trocar system typically includes a pointed rod-like device or obturator fitted into a tube-like device or cannula. The cannula head of the cannula often has one or more seals. A pointed end of the obturator projects out an end of a cannula tube and is used to penetrate the outer tissue of the cavity. After the tissue is penetrated and the body cavity is accessed by the trocar system, the obturator is then withdrawn while the cannula tube is retained in the cavity. The body cavity can then be accessed by surgical instruments via the cannula tube to perform various surgical procedures, or the cannula can simply be used as a drainage outlet.

There is a need for improved seals for trocar systems which allow a surgeon to provide an opening in a cavity of a patient and though which surgical instruments may be inserted.

### SUMMARY OF THE INVENTION

In one aspect, the present invention provides a seal for a cannula head in which the seal includes a support defining a passageway extending therethrough, a flexible seal supported by the support across the passageway, and a ring disposed between a lower portion of the support and the flexible seal, and wherein the ring defines an opening sized smaller than an opening in the lower portion of the support. The ring may be movable or fixed in position between the flexible seal and the lower portion of the support. The opening in the ring is sized smaller than the opening in the lower support.

In another aspect, the present invention provides cannula heads having a housing with a passageway extending therethrough and the above-described seal disposed across the passageway.

In a further aspect, the present invention provides a method for sealing the passageway of a cannula head in which the method includes providing a support sealably insertable across the passageway. The support has a substantially central opening substantially aligned with the passageway and terminating distally in a lower opening. A flexible seal is sealably attaching above the lower opening across the central opening of the support, and a ring is positioned within the central opening of the support between the lower opening of the support and the flexible seal. The ring is larger in size than the lower opening and having an inner opening sized smaller than the lower opening of the support.

### BRIEF DESCRIPTION OF THE DRAWINGS

The subject matter which is regarded as the invention is particularly pointed out and distinctly claimed in the concluding portion of the specification. The invention, however, may best be understood by reference to the following detailed descriptions of the various embodiments and the accompanying drawings in which:

FIG. 1 is a perspective view of a dilating trocar system in accordance with the present invention;

FIG. 2 is a perspective view of a cutting trocar system in accordance with the present invention;

FIG. 3 is a side elevational view, in part cross-section, of the dilating trocar system of FIG. 1 showing the obturator removed from the cannula;

FIG. 4 is a side elevational view, in part cross-section, of the cutting trocar system of FIG. 2 showing the obturator removed from the cannula;

FIG. 5 is an enlarged, exploded, cross-sectional view of the cannula of FIGS. 1 and 2 and a releasably attachable upper seal in accordance with the present invention;

FIG. 6 is an enlarged, exploded, cross-sectional view of the cannula head of FIGS. 1 and 2 showing a lower seal with a flapper valve open;

FIG. 7 is an exploded, perspective view of the lower seal of FIG. 6;

FIG. 8 is an exploded, cross-sectional view of the lower seal of FIG. 6;

FIG. 9 is an enlarged perspective view, partially cutaway, of the releasably attachable upper seal of FIG. 5;

FIG. 10 is a top view of the protective covering guide of the releasably attachable upper seal shown in FIG. 9;

FIG. 11 is a cross-sectional view of the releasably attachable upper seal of FIG. 9;

FIG. 12 is a cross-sectional view of another embodiment of a releasably attachable upper seal in accordance with the present invention;

FIG. 13 is a cross-sectional view of another embodiment of a releasably attachable upper seal in accordance with the present invention;

FIG. 14 is a top view of a cannula head with a releasably attachable upper seal shown in dashed lines in an unlocked position;

FIG. 15 is a top view of a cannula head with a releasably attachable upper seal shown in a locked position;

FIG. 16 is a cross-sectional view of a releasably attachable upper seal with an obturator extending therethrough; and

FIG. 17 is a cross-sectional view of a releasably attachable upper seal with an obturator off centered extending therethrough.

### DETAILED DESCRIPTION OF THE INVENTION

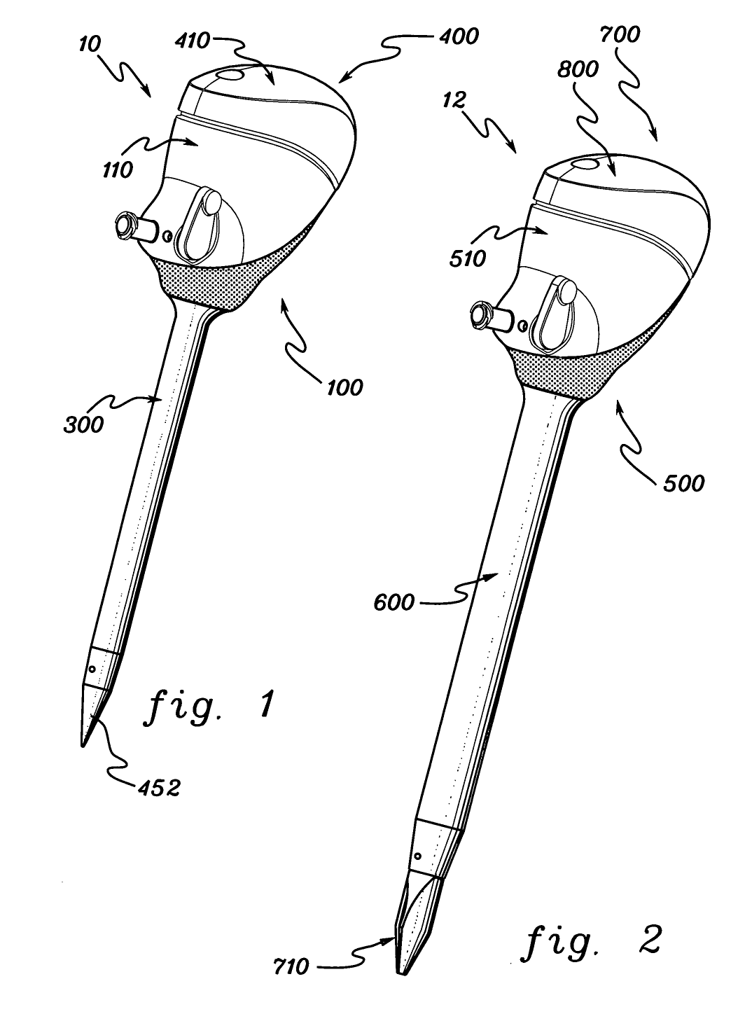

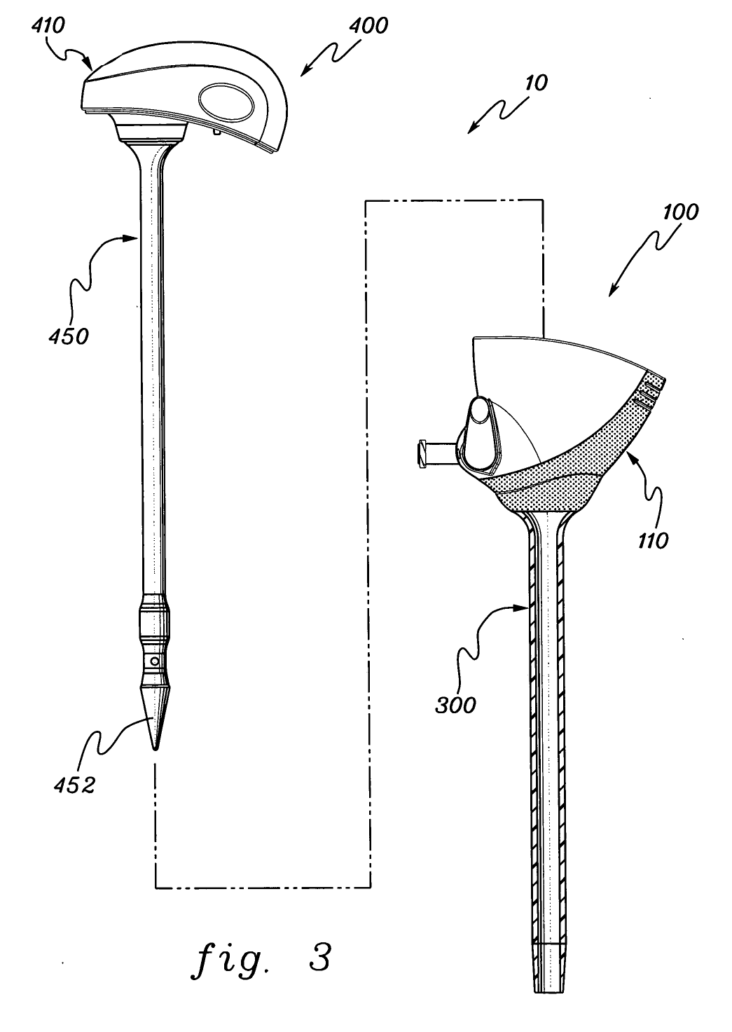

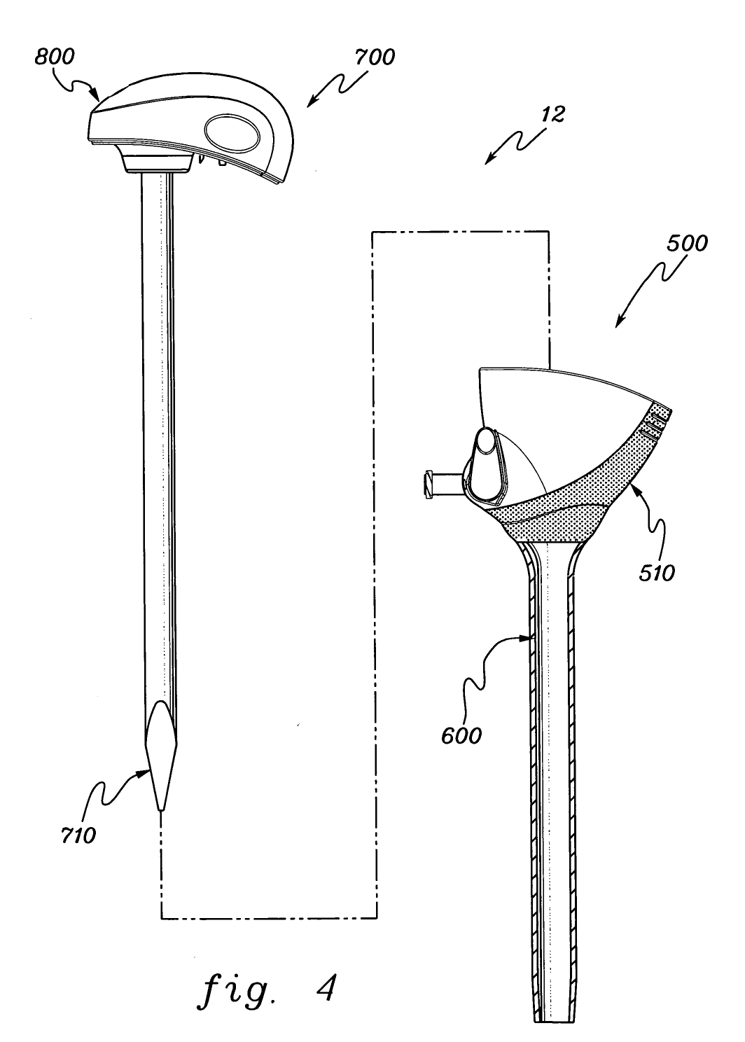

FIGS. 1 and 2 illustrate perspective views of a dilating trocar system 10 and a safety-shielded cutting trocar system 12, respectively, which may incorporate a seal in accordance with the present invention as explained in greater detail below. With reference to FIGS. 1 and 3, dilating trocar system 10 generally includes a cannula 100 having a cannula head 110 and a cannula tube 300 into which is slidably receivable a dilating obturator 400 having an obturator cap 410 attached to an elongated shaft 450 (FIG. 3) having a pointed end 452 which may have a rounded point at its distal end. With reference to FIGS. 2 and 4, trocar system 12 generally includes a cannula 500 having a cannula head 510 and a cannula tube 600 into which is slidably receivable a safety shielded cutting obturator 700 having a cap 800 and a spring-loaded shield 710 and automatically-operated spring-loaded locking mechanism for inhibiting the exposure of a cutting blade (not shown) after passing through tissue or muscle. The cutting trocar system cuts or lacerates tissue when obtaining access to a body cavity. The dilating trocar systems allow parting and stretching of, for example, multiple cross-directional muscle layers and intra-abdominal blood vessels, when gaining access to a body cavity.

The above-noted trocar systems may include one or more of the various features described in U.S. patent application Ser. No. 10/264,530, filed Oct. 4, 2002, entitled “Trocar System,” the entire subject matter of which is incorporated herein by reference.

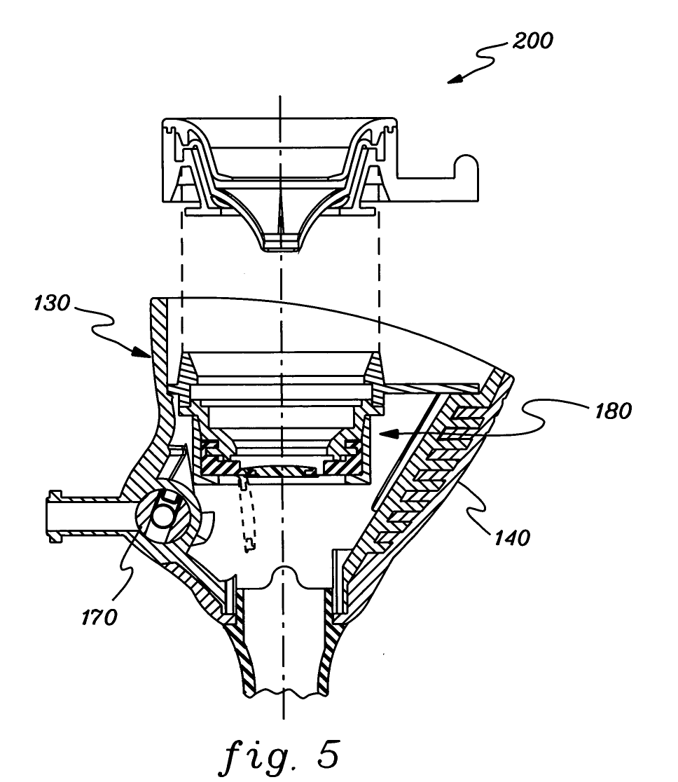

As shown in FIG. 5, the cannulas may include a cannula housing 130, resilient non-slip material 140, a releasably attachable stopcock valve 170, a lower seal 180, and a releasably attachable upper seal 200.

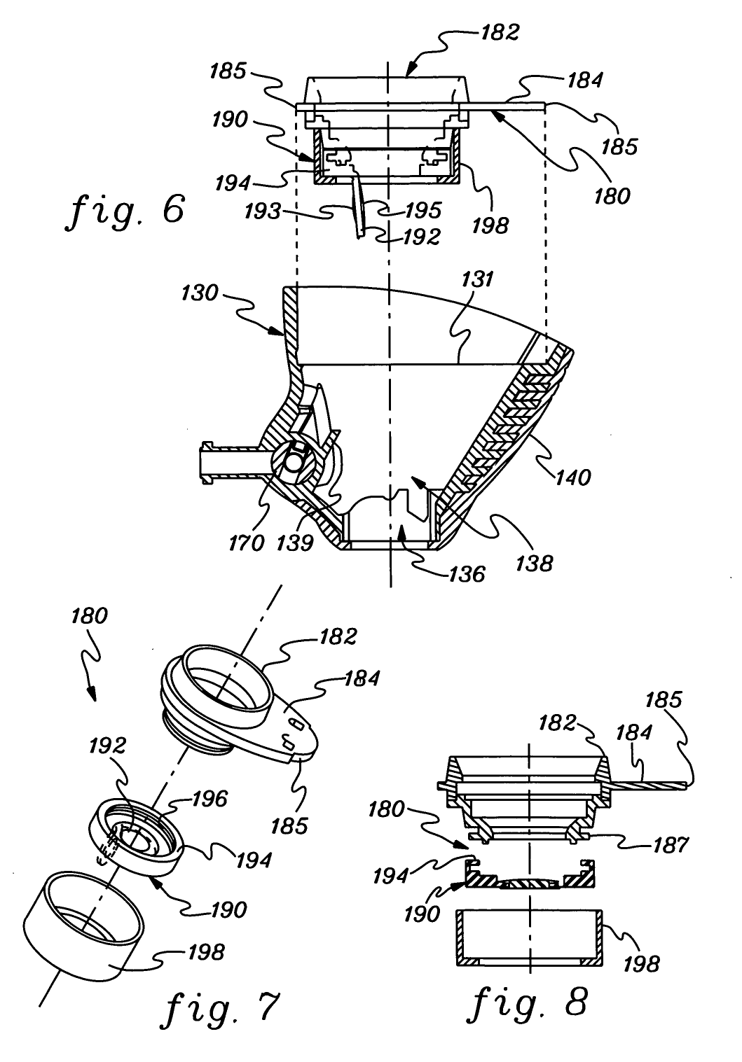

As best shown in FIGS. 6-8, lower seal 180 includes an outer support 182, a flapper valve 190, and a retainer cap 198. Lower seal 180 is sealably attached across passageway 136 (FIG. 6) in cannula housing 130 (FIG. 6) behind stopcock valve 170 (best shown in FIG. 5). For example, an outer edge 185 of a lateral flange 184 may be attached with an adhesive (or other suitable means for forming a complete seal) along a support or ledge 131 (FIG. 6) formed in cannula housing 130 (FIG. 6) to define a chamber 138 (FIG. 6) in housing 130 (FIG. 6).

Flapper valve 190 includes a flexible disc-shaped portion 192 (shown in an open position in FIG. 6) attached at a portion along its circumference to a flexible collar 194 having a groove 196 (FIG. 7) therein. A pair of rigid discs 193 and 195 (best shown in FIG. 6) may be attached to the center of flexible disc-shaped portion 192. The rigid discs may add support to and protect the flexible disc-shaped portion when an obturator or other instruments are inserted and removed from the cannula. Outer support 182 may be monolithic or integrally formed as one-piece. Outer support 182 may also include a circumferentially extending flange 187 (best shown in FIG. 8) on which is received flexible collar 194 of flapper valve 190. When no instrument is inserted in the cannula, the flapper valve 190 is normally closed, i.e., biased shut. In addition, if the cannula housing is pressurized with a fluid, the pressurized fluid will exert a pressure against the flapper valve to more securely retain the flapper valve in a sealed position and maintain the pressure in the cannula housing and in the body cavity. Retainer cap 198 further secures and maintain flapper valve on outer support 182. In particular, retainer cap 198 aids in retaining collar 194 (FIG. 6) or flapper valve 190 (FIG. 6) on outer support 182 (FIG. 6) when an obturator is inserted in the cannula. Retainer cap may include one or more notches or cutouts (not shown) on the lower portion thereof adjacent to where the flapper valve attaches to flexible collar 194. This notch or notches allow the hinge of the flapper valve to be more flexible and seat properly if caught by a hooked instrument as it is withdrawn from the cannula.

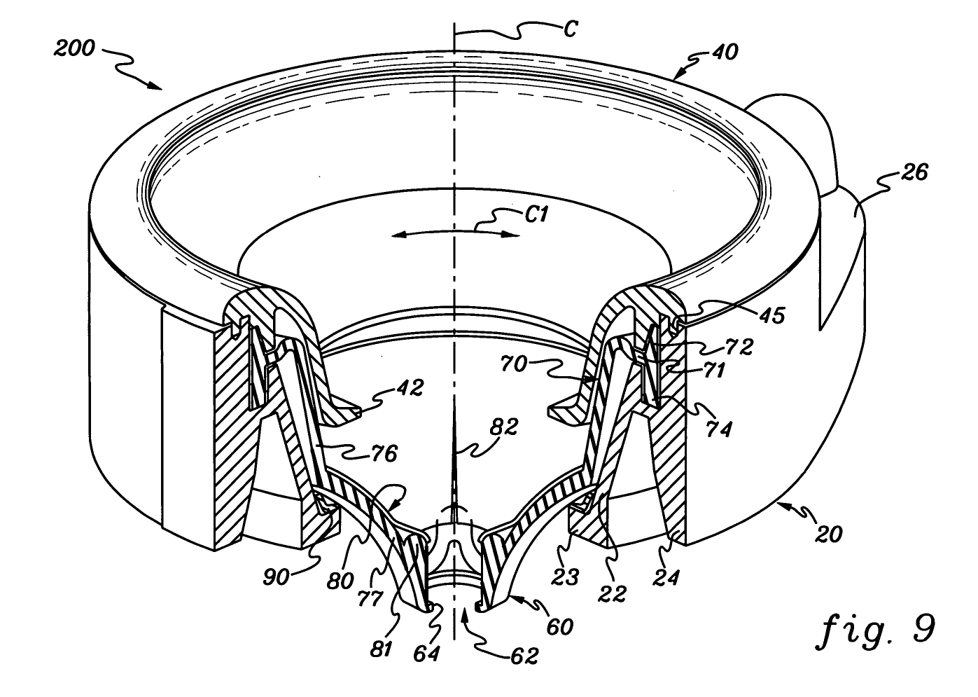

As shown in FIG. 9, releasably attachable upper seal 200 may include a rigid distal support 20, a rigid proximal support 40, a rotatable flexible seal 60 having upper portions sandwiched between the supports, a movable ring 90 disposed between the flexible seal and the lower support, and a generally non-compliant, but bendable protective guide 80. As described above, upper seal assembly 200 is releasably and sealably attachable to the cannula head of the trocar as illustrated in FIG. 5.

With reference again to FIG. 9, distal support 20 includes an inner ring-shaped portion 22 having an inwardly-extending flange 23 defining a distal opening, an outer ring-shaped portion 24, and an outwardly-extending arm 26. Outwardly extending arm 26 allows a surgeon to readily lock and unlock upper seal assembly 200 to a cannula head, e.g., via a bayonet-style connector as described in greater detail below. This locking and unlocking arrangement permits a surgeon to remove upper seal 200 if it becomes necessary during surgery to withdraw a tissue sample or another sizeable item from the surgical site. Otherwise, upper seal 200 generally remains in place during a surgical procedure because it can accept instruments having various diameters.

Rigid proximal support 40 includes an inwardly-extending flange 42 which defines a proximal opening. The openings in distal support 20 and proximal support 40 guide and limit lateral movement of an instrument inserted in upper seal assembly 200. Proximal support 40 may also have a concave shape for directing a tip of an instrument through the upper seal. The distal and proximal supports may be made from a rigid plastic material.

Rotatable flexible seal 60 includes a distal centrally located aperture or opening 62 which allows for easy insertion and removal, for example, of an instrument such as an obturator shaft into a cannula head while inhibiting the release of fluid from the cannula head. The distal end of rotatable flexible seal 60 may also include and inwardly extending flange 64 which assists in conforming and sealing against relatively small instruments but which flattens out against and conforms to relatively large instruments. The distalmost portion of rotatable flexible seal 60 also includes a raised sinusoidal portion 81 that extends around the inner, lower portion of the upper seal to define a plurality of inwardly extending lobes. This configuration allows the opening to easily expand and conform around relatively large instruments.

Rotatable flexible seal 60 further includes an upper seal portion 70 having a horizontally-extending portion 71, an upwardly-extending annular flange 72, and a downwardly-depending annular flange 74. The flanges 72 and 74 and a portion of the horizontally-extending portion 71 are fitted between portions of proximal support 40 and an annular groove in distal support 20. An outer downwardly-depending flange 45 of proximal support 40 may be received in and fixedly attached, e.g., with adhesive or by ultrasonic welding, to an annular groove in distal support 20.

Upper seal portion 70 of the rotatable flexible seal is sized slightly smaller than the corresponding cutout formed in the rigid supports. Accordingly, this allows the seal to rotate when an instrument is inserted in and twisted about the central axis C of the seal in the direction of curved double-headed arrow C1. In addition, the pressurized cannula results in forcing the outer surface of downwardly-depending annular flange 74 against the inside surface of the cutout formed in rigid support 20 to form an airtight seal to seal pressurized gas in the cannula head and prevent leakage.

Disposed between upper seal portion 70 and opening 62 is a generally downwardly-depending vertical frusta conical wall portion 76 and a convex wall portion 77. Rotatable flexible seal 60 may be made from a resilient or an elastomeric material, for example, a silicone rubber, a polyurethane elastomer, a neoprene or a thermoplastic elastomer. A lubricant may be applied to the outer surface of the upper seal 70 (e.g., to the upper flange portion) or to the entire surface of the upper seal 70. The lubricant may be a coating, such as parylene.

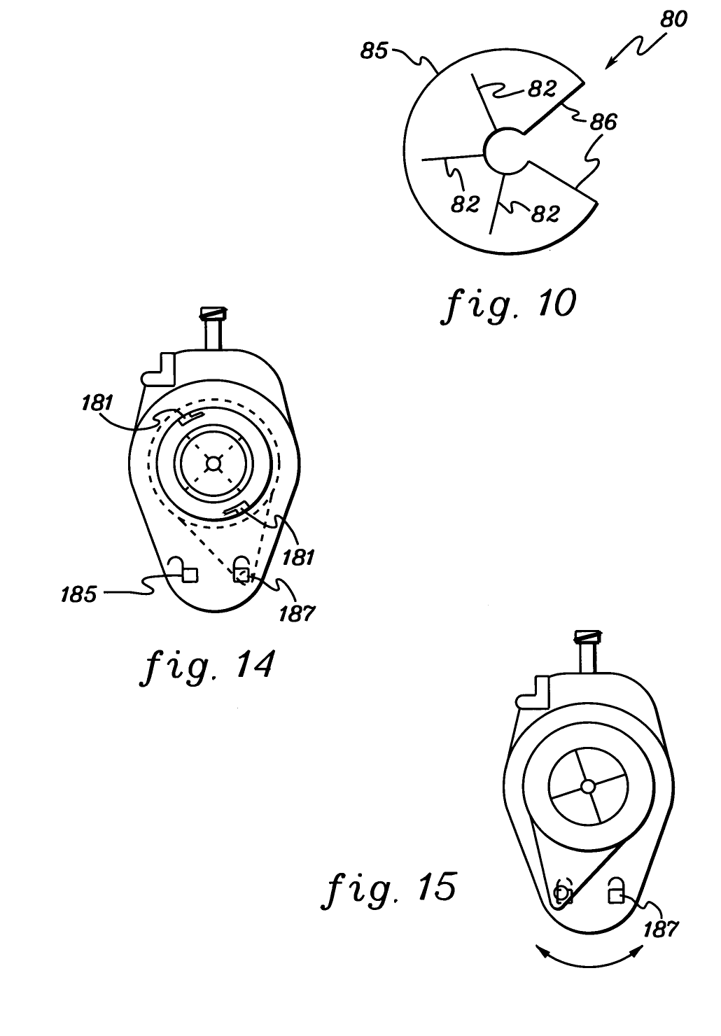

Protective guide or liner 80 is attached, for example with an adhesive, along an inner surface of convex seal portion 77. A lower portion of the protective guide is spaced apart from or disposed away from opening 62 in rotatable flexible seal 60 in the vicinity of the lobes, short of orifice 62, thus leaving a portion of seal 60 exposed for making a seal with an instrument. Protective guide further includes four slits 82, only one of which is shown in FIG. 9. As best shown in FIG. 10, protective guide 80 may be fabricated from a flat sheet initially in the form of an arc such as from a slippery, bendable material such as a thin layer or sheet of plastic. Slits 82 are formed extending from an inner portion 84 to a point adjacent to an outer edge of an outer portion 85. Accordingly, when protective guide 80 is attached to the inner surface of convex portion 77 (FIG. 9), edges 86 are disposed adjacent to each other. The slits in the guide permit the opening in the guide to expand radially outward as the width of each slit widens upon insertion of an instrument.

As best shown in FIG. 11, a movable washer 90 may be disposed between rotatable flexible seal 60 and flange 23. The purpose of washer 90 is to restrain the distal movement of flexible seal 60 when an instrument of large diameter is inserted therein and to inhibit the rotatable flexible seal 60 from being stretched distally to such an extent that it becomes jammed at the orifice of the lower flapper valve. Movable washer 90 may be made to generally replicate the shape and the dimensions of flange 42 of proximal support 40. For example, the washer may have a curved cross-section. Thus, the central opening in washer 90 may essentially match that of the central opening of flange 42. Washer 90 may be fabricated from a rigid plastic material such as Acrylonitrile Butadiene Styrene (ABS), polycarbonate, nylon, etc.

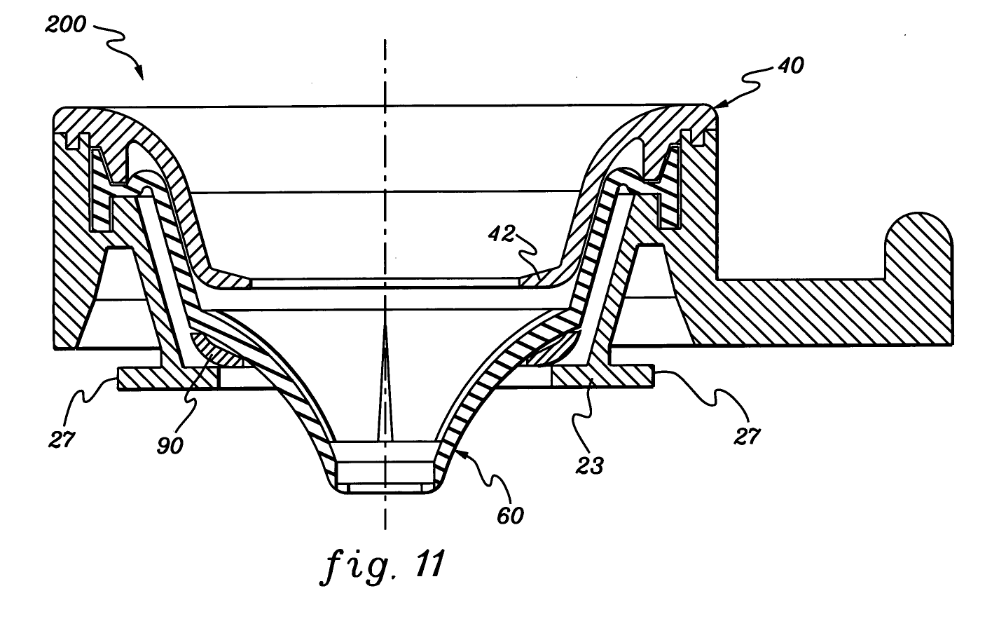

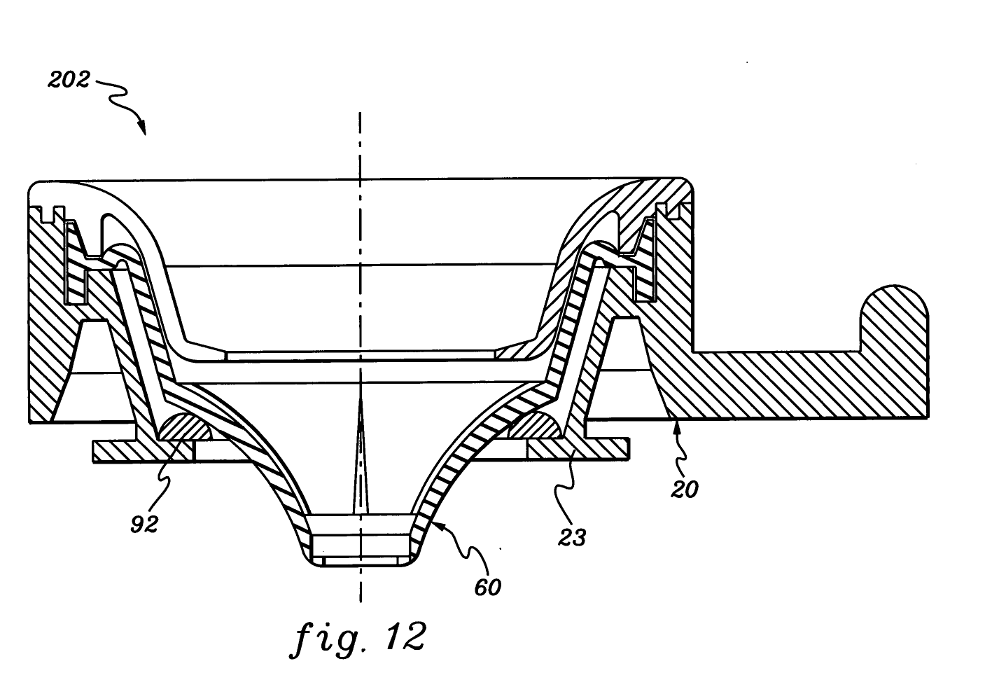

FIG. 12 illustrates another embodiment of releasably attachable upper seal 202 in accordance with the present invention. In this embodiment, a ring 92 configured as half of an O-ring (e.g., having a half round cross-section) may be disposed between rotatable flexible seal 60 and flange 23. Ring 92 may be formed from a resilient material such as silicone rubber or other suitable material, and may be fixedly attached to flange 23 with an adhesive.

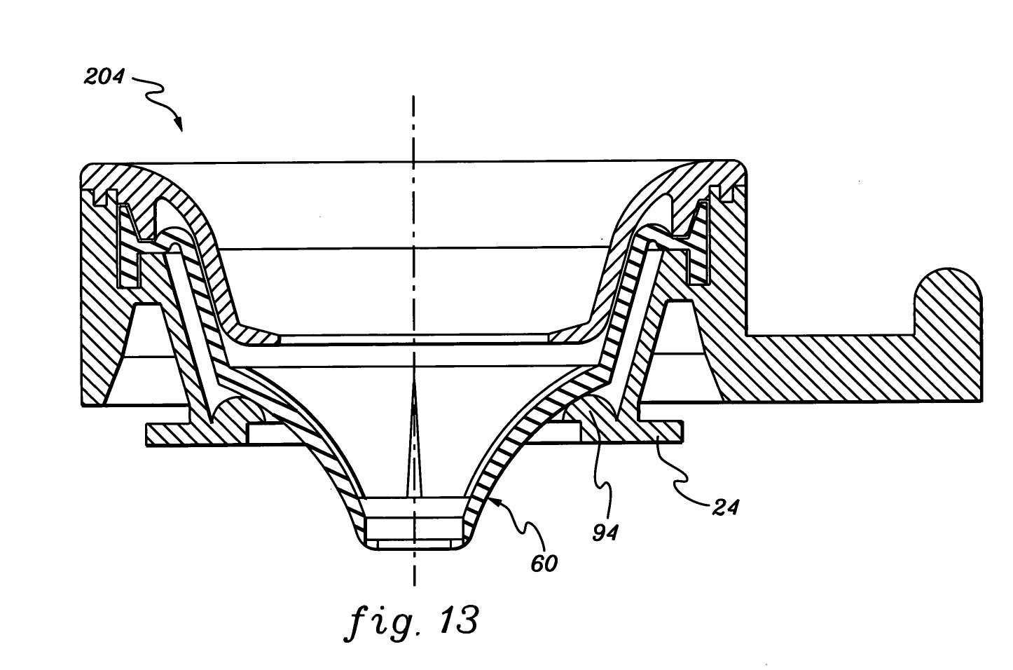

FIG. 13 illustrates another embodiment of releasably attachable upper seal 204 in accordance with the present invention. In this embodiment, a flange 24 of rigid distal support 20 is configured to include a raised portion 94 which extends upwardly and inwardly toward flexible seal 60. Thus, raised portion 94 may be integrally formed as a unit with distal support, i.e., being monolithic or as one piece.

For example, the proximal opening defined by flange 42 may measure about ½ of an inch in diameter and the orifice in the seal may measure about ⅛ of an inch in diameter. Flange 23 on distal support 20 may define a distal opening measuring about ⅝ of an inch in diameter. The washer opening may measure about ½ of an inch in diameter.

As shown in FIG. 14, a releasable attachable seal is shown in dashed lines in an unlocked position. As shown in FIG. 15, the releasably attachable seal is releasably attached to the cannula head by rotating the releasably attachable seal clockwise.

The cannula head and the releasably attachable seal may be releasably attachable, for example, via a bayonet-type connector, with the releasably attachable seal having two flexible pins or tabs 27 (FIG. 11) that extend outwardly and engage J-shaped grooves 181 (FIG. 14) in the lower seal of the cannula head. It will be appreciated that other means for releasably attaching the upper seal to the cannula head may be employed.

In addition, indicia 185 and 187 may be provided on the lower seal for indicating the locked and unlocked positions of the releasably attachable upper seal. By providing a releasably attachable upper seal, a surgeon may remove the releasably attachable upper seal to provide a greater opening for removing, for example, tissue through the cannula tube and cannula head. The aperture of the upper seal may be sized so that the aperture or hole is slightly smaller than an obturator shaft or surgical instrument to be used. This interference fit between the resilient diameter of the aperture and the shaft minimizes the passage of fluid from a cavity to the ambient environment during insertion and removal of an obturator or other instruments. When an obturator, for example, is removed from the cannula and from the aperture, the flapper valve provides a seal to the ambient environment.

From the present description, it will be appreciated that the cannula head may have a fixedly attached upper seal. In addition, the releasably attachable upper seal may include a stationary ring or fixedly attached ring while the flexible seal is operable to rotate within the supports. In addition, the releasably upper seal may include a nonrotatable flexible seal and a movable or fixedly attached ring. It will also be appreciated that the ring may have other suitable cross-sectional configurations.

It will be appreciated by those skilled in the art that the diameter of the opening in the proximal support, the thickness of the flexible seal, and the diameter of the aperture in the flexible seal, may vary depending upon the size of the cannula head, the size of the obturator or other instruments to be used, and the difference in pressure across the flexible seal that needs to be sealed. For example, the thickness of the flexible seal may be between about 1 mm to about 3 mm, and the thickness of the protective guide may be about 0.010 inches to about 0.030 inches.

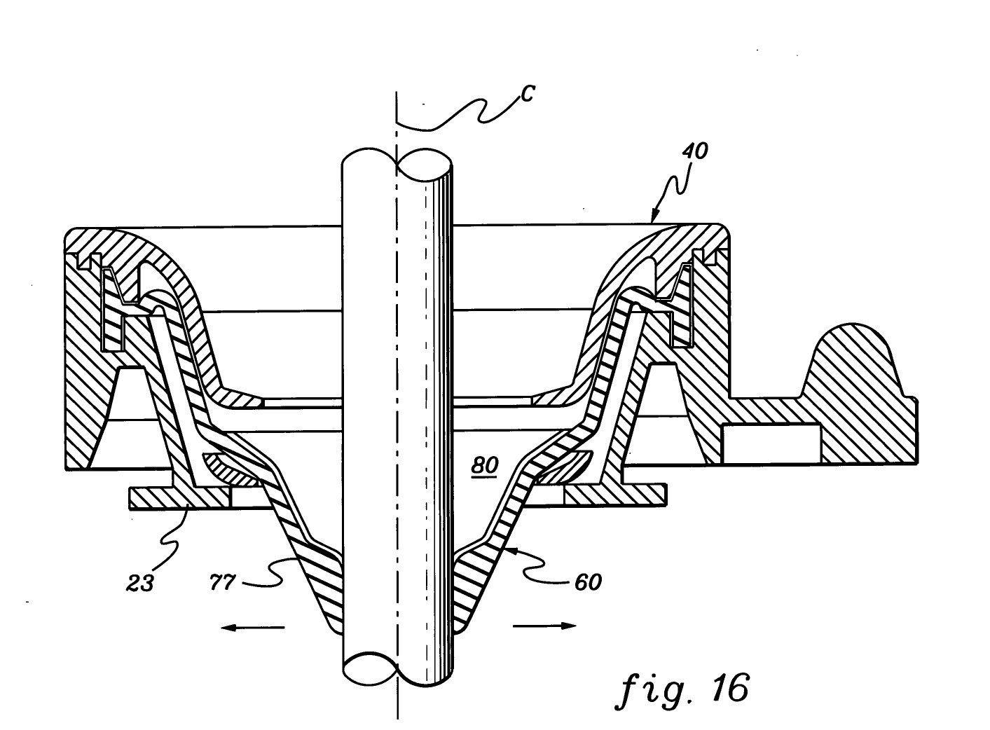

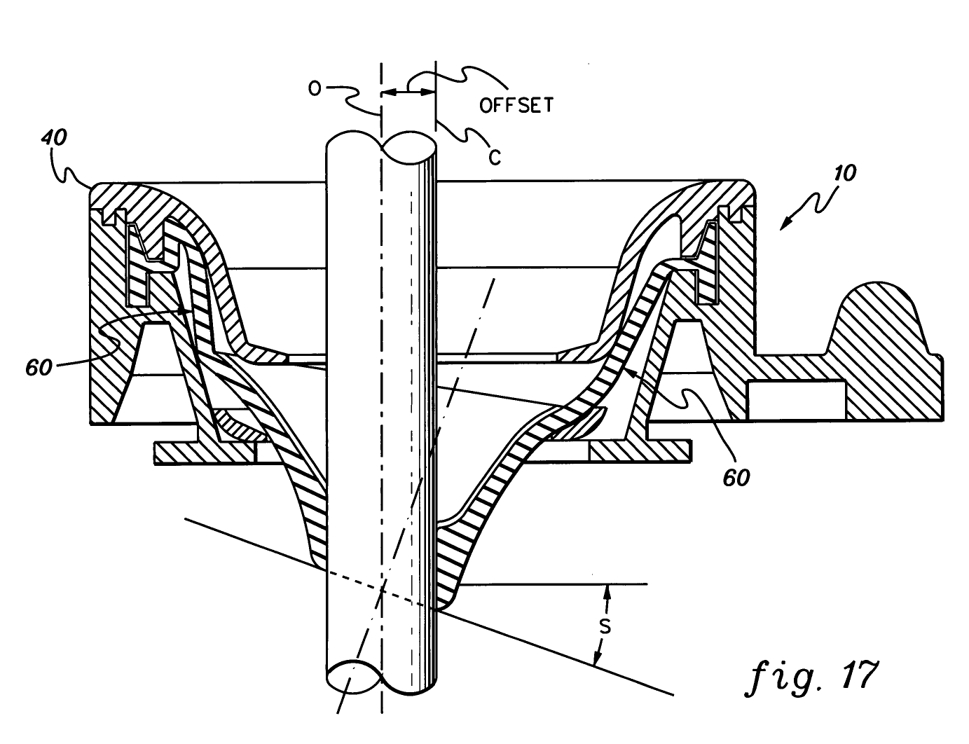

With reference to FIGS. 16 and 17, when assembled, the releasably attachable upper seal is designed primarily so that any lateral movement of the seal 60 will be rather limited when an instrument is initially inserted off axis. Instead, the instrument is guided or deflected toward the central axis C. In particular, the combination the dish-shaped proximal support 40, the funnel or conical shape of rotatable flexible seal 60, the conical shape and slipperiness of the protective guide 80 which extends generally over the portion of the flexible seal exposed under the proximal opening formed in the proximal support 40, the washer, and the lower flange, all result in a distal point of an instrument being guided or deflected toward the central opening of the flexible seal.

As shown in FIG. 16, an instrument such as a shaft of an obturator is ideally inserted along central axis C of the releasably attachable upper seal. As the point of an obturator or instrument is inserted, the opening of the seal 60 expands in the direction of the horizontal arrows to form a seal around its shaft. Under normal circumstances, an instrument inserted off axis will simply be deflected by protective guide 80 toward the central axis C.

As best shown in FIG. 17, if a relatively large force is applied and maintained to hold the off axis (e.g., along axis O) once the shaft of the obturator is fully inserted in the releasably attachable upper seal, the combination of flexible seal 60 and protective guide 80 deforms and tilts resulting in the lower portion thereof maintaining a seal around the shaft.

In particular, the upper portion of flexible seal 60 closest to the shaft is deformed and pushed upwardly against proximal support 40,.and the upper portion of flexible seal 60 farthest away from the shaft is pulled downwardly. In addition, the combination of the flexible seal 60 and protective guide 80 are deformed and disposed on an angle to central axis C and the lowermost portion of flexible seal 60 is deformed and disposed on an angle S around shaft 100. The lowermost portion forming the resulting opening in flexible seal 60 around the shaft is no longer circular but instead oval shaped.

While the invention has been particularly shown and described with reference to preferred embodiment, it will be understood by those skilled in the art that various changes in form and details may be made to the invention without departing from the spirit and scope of the invention described in the following claims.

1. A seal for a cannula head, said seal comprising:
a support defining a passageway extending therethrough;
a flexible seal supported by said support across said passageway;
a ring disposed between a lower portion of said support and said flexible seal; and
wherein said ring defines an opening sized smaller than an opening in said lower portion of said support.

2. The seal of claim 1 wherein said ring is movable between said flexible seal and said lower portion of said support.

3. The seal of claim 1 wherein said ring is fixedly attached to an inwardly-extending flange of said lower portion of said support.

4. The seal of claim 1 wherein said ring is integrally formed with an inwardly-extending flange of said lower portion of said support so that said ring is disposed upwardly and inwardly from said inwardly-extending flange.

5. The seal of claim 1 wherein an upper portion of said support comprises an upper opening which corresponds in size to said opening in said ring.

6. The seal of claim 1 wherein said ring comprises a curved cross-section.

7. The seal of claim 1 wherein said ring comprises a half round cross-section.

8. The seal of claim 1 wherein said ring comprises a rigid material.

9. The seal of claim 1 wherein said ring comprises a resilient material.

10. The seal of claim 1 wherein said seal comprises a guard having a plurality of slits for protecting said flexible seal.

11. The seal of claim 1 wherein said flexible seal is rotatably supported by said support.

12. The seal of claim 11 wherein said flexible seal comprises an upper portion having a horizontally-extending portion, and an upwardly-extending annular flange, and a downwardly-depending annular flange.

13. The seal of claim 1 wherein said seal is releasably attachable to the cannula head.

14. The seal of claim 1 wherein at least one of said support comprises an arm outwardly extending from said support.

15. The seal of claim 14 wherein said arm comprises an upwardly-extending knob.

16. A cannula head comprising:
a housing having a passageway extending therethrough; and
a seal of claim 1 disposed across said passageway;

17. The cannula head of claim 16 wherein said seal is releasably attachable to said cannula head.

18. A trocar system comprising:
a cannula head comprising a housing having a passageway extending therethrough;
a seal of claim 1 disposed across said passageway;
an obturator receivable in said cannula head and through said seal; and
a cannula tube attachable to said cannula head.

19. The trocar system of claim 18 wherein said seal is releasably attachable to said cannula head.

20. A method for sealing the passageway of a cannula head, the method comprising:
providing a support sealably insertable across the passageway, the support having a substantially central opening substantially aligned with the passageway and terminating distally in a lower opening;
sealably attaching above the lower opening a flexible seal across the central opening of the support; and
positioning a ring within the central opening of the support between the lower opening of the support and the flexible seal, the ring being larger in size than the lower opening and having an inner opening sized smaller than the lower opening of the support.

21. The method of claim 20 wherein the ring is floatably positioned between the flexible seal and the support.

22. The method of claim 20 further comprising fixedly attaching the ring to an inwardly-extending flange of the support.

23. The method of claim 20 further comprising integrally forming the support and the ring as one piece.

24. The method of claim 20 wherein the attaching step comprises rotatably attaching the flexible seal to the support.

25. The method of claim 20 wherein the support is fixedly inserted into the cannula head.
